(19) United States
(12) Patent Application Publication
(10) Pub. No.: US 2017/0043158 A1
(43) Pub. Date: Feb. 16, 2017

(54) INTERVENTIONAL MEDICAL SYSTEMS AND CATHETERS

(71) Applicant: Medtronic, Inc., Minneapolis, MN (US)

(72) Inventors: Tomas K. Kelly, Galway (IE); Brendan P. Geraghty, Galway (IE); Paula McDonnell, Galway (IE); Francis D. McEvoy, Laois (IE); Pat McHugh, Ballyhaunis Co. Mayo (IE); Colin W. Meade, Westmeath (IE); Kealan E. O'Carroll, Galway (IE); Rónán Wood, Galway (IE); Michael B. Sayers, Kerry (IE)

(21) Appl. No.: 15/332,373

(22) Filed: Oct. 24, 2016

Related U.S. Application Data
(63) Continuation-in-part of application No. 15/234,023, filed on Aug. 11, 2016, now abandoned.
(60) Provisional application No. 62/204,235, filed on Aug. 12, 2015.

(51) Int. Cl. A61N 1/05; A61M 25/01

(52) U.S. Cl. CPC A61N 1/0573; A61M 25/0147; A61N 2001/0578

(57) ABSTRACT

An interventional medical system includes an elongate shaft, a steering assembly coupled to the shaft, a snare member, and a passageway configured to allow passage of the snare member therethrough and into a first longitudinally extending lumen of the shaft; wherein the passageway is spaced distally from the steering assembly actuator by no more than 3 cm to 3.5 cm, and the passageway includes an elastic sealing perimeter. The passageway may, in some cases, be formed in a tapered member of the steering assembly that couples a proximal end of the shaft to a core of the steering assembly.

### CROSS-REFERENCE TO RELATED APPLICATION

The present application claims priority to U.S. Provisional Patent Application Ser. No. 62/204,235, which was filed on Aug. 12, 2015, and U.S. patent application Ser. No. 15/234,023, which was filed on Aug. 11, 2016, both are incorporated by reference in their entirety.

### FIELD OF THE DISCLOSURE

The present disclosure pertains to interventional medical systems, and more particularly to those that are useful for retrieving medical devices from implant sites.

### BACKGROUND

The traditional implantable cardiac pacemaker includes a pulse generator device to which one or more flexible elongate lead wires are coupled. The device is typically implanted in a subcutaneous pocket, remote from the heart, and each of the one or more lead wires extends therefrom to a corresponding electrode, coupled thereto and positioned at a pacing site, either endocardial or epicardial.

Mechanical and/or MRI compatibility issues, which are sometimes associated with elongate lead wires and well known to those skilled in the art, have motivated the development of implantable cardiac pacing devices that are wholly contained within a relatively compact package, the entirety of which is configured for implant in close proximity to the pacing site. FIG. 1 is a schematic diagram that shows potential cardiac implant sites for such a device, for example, within an appendage 102 of a right atrium RA, within a coronary vein CV (via a coronary sinus ostium CSOS), or in proximity to an apex 103 of a right ventricle RV, for example, as shown in FIG. 2.

FIG. 2 shows an exemplary implantable medical device 300 having been implanted by an operator using a catheter/tool 200, for example, like that described in the commonly assigned United States Patent Application US 2015/0094668, wherein the operator advanced tool 200 into the right heart through the inferior vena cava IVC, for example, from a femoral vein access site, and then deployed device 300 from a receptacle defined by a distal-most portion 230 of tool 200. In some cases, when it may be necessary to retrieve the implanted device, the operator can employ tool 200 to do so, but new and improved tools and methods would increase the ease and efficiency of retrieval.

### BRIEF SUMMARY

Embodiments and methods disclosed herein facilitate the use of a snare member to retrieve an implanted medical device. According to some embodiments, an interventional medical system includes an elongate shaft, a steering assembly coupled to the shaft, a snare member, and a passageway configured to allow passage of the snare member therethrough and into a first longitudinally extending lumen of the shaft; wherein the passageway is spaced distally from the steering assembly actuator by no more than 3 cm to 3.5 cm, and the passageway includes an elastic sealing perimeter. The passageway may, in some cases, be formed in a tapered member of the steering assembly that couples a proximal end of the shaft to a core of the steering assembly.

### DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments will hereinafter be described in conjunction with the appended drawings wherein like numerals denote like elements, and:

FIG. 1 is a schematic diagram showing potential implant sites for a relatively compact implantable medical device;

FIG. 2 is a schematic diagram showing an exemplary relatively compact implantable medical device having been delivered from a catheter to an implant site;

FIG. 3 is a plan view of the exemplary relatively compact implantable medical device, which may be part of an interventional medical system, according to some embodiments;

FIG. 4A is a plan view of a catheter and a retrieval tool that may be used together in an interventional medical system, according to some embodiments;

FIG. 4B is a perspective view of the catheter and retrieval tool engaged together to form the interventional medical system, according to some embodiments;

FIG. 4C is plan view, with an enlarged detail, and with cut-away cross-sections, of the catheter of FIGS. 4A-B, according to some embodiments;

FIG. 5A is a plan view of an interventional medical system according to some alternate embodiments;

FIG. 5B is a cross-section view through a portion of a retrieval tool of the system of FIG. 5A, according to some embodiments; and

FIGS. 6A-F are schematics outlining some methods of the present invention.

### DETAILED DESCRIPTION

The following detailed description is exemplary in nature and is not intended to limit the scope, applicability, or configuration of the invention in any way. Rather, the following description provides practical examples, and those skilled in the art will recognize that some of the examples may have suitable alternatives. The term “approximately,” as used in this specification and appended claims, refers to plus or minus 5% of the value given.

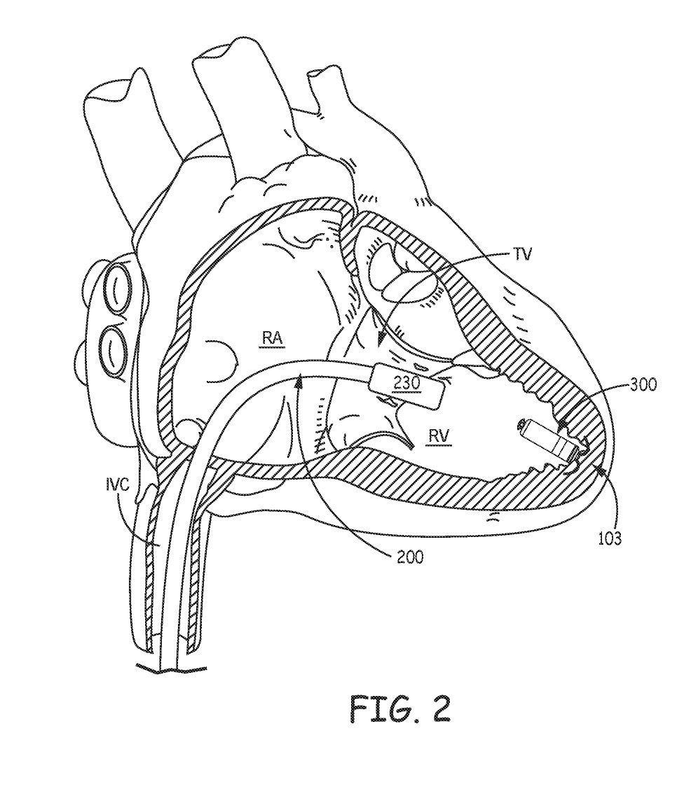

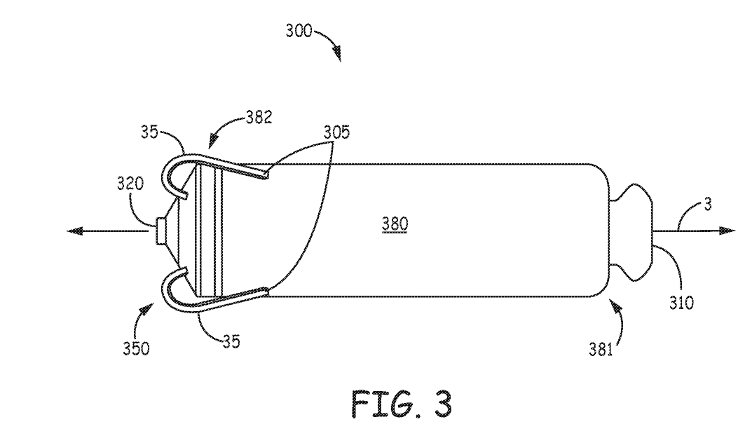

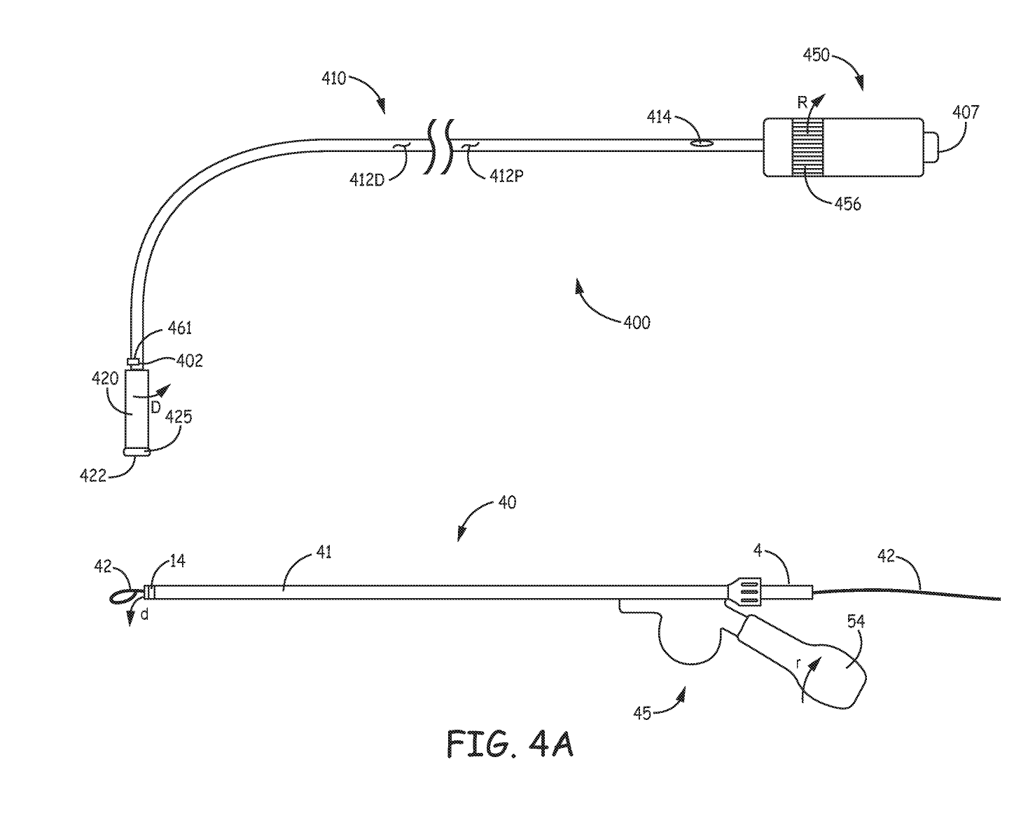

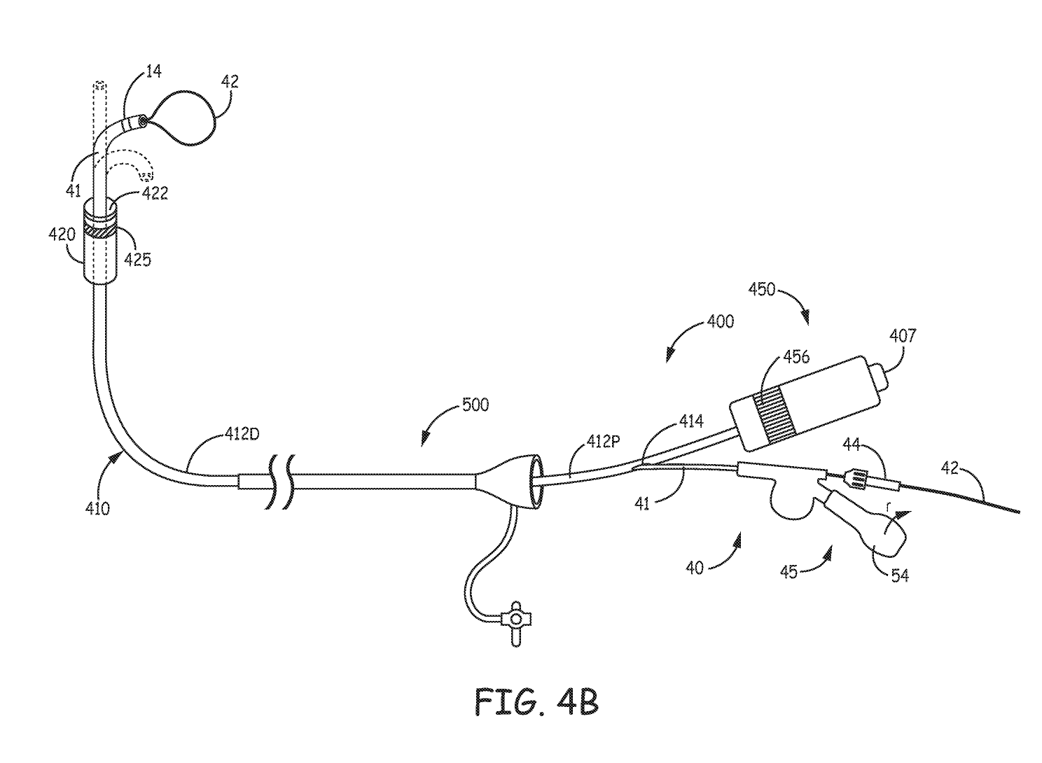

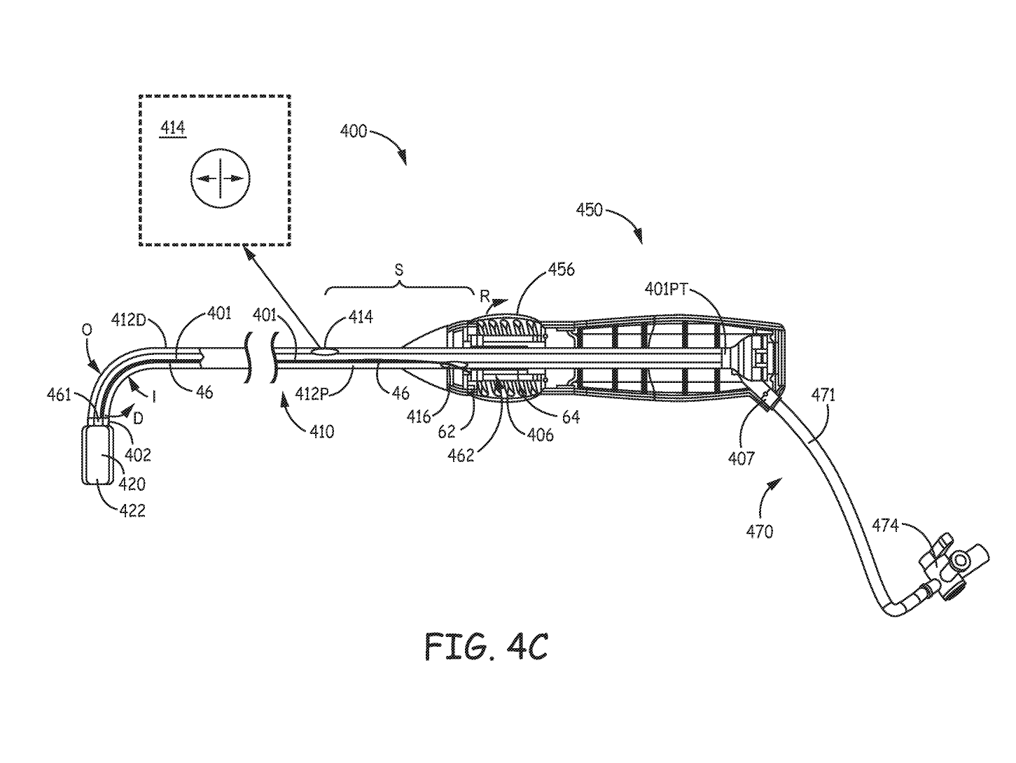

FIG. 3 is a plan view of the exemplary relatively compact implantable medical device 300 from FIG. 2, which may be part of an interventional medical system, according to some embodiments. FIG. 3 illustrates device 300 including a hermetically sealed housing 380 extending from a proximal end 381 thereof to a distal end 382 thereof and along a longitudinal axis 3. Device 300 further includes an electrode 320 and a fixation member 350, both mounted in proximity to distal end 382 of housing 380, and an electronic controller (not shown), for example, a pulse generator and an associated power supply, contained in housing 380, wherein electrode 320 is electrically coupled to the controller via a hermetically sealed feedthrough assembly (not shown) such as is known in the art. Housing 380, for example, formed from a biocompatible and biostable metal such as titanium, may be overlaid with an insulative layer, for example, medical grade polyurethane, parylene, or silicone, and, although not shown, device 300 may include another electrode, for example, formed by removing a portion of the insulative layer to expose the metallic surface of housing 380. The other electrode may function in conjunction with electrode 320 for bipolar pacing and sensing, when fixation member 350 secures electrode 320 in intimate tissue contact at a target implant site. FIG. 3 further illustrates device 300 including an attachment feature 310 joined to proximal end 381 of housing 380, wherein feature 310 is configured for snaring, for example, by a snare member 42 described below in conjunction with FIGS. 4A-B and 5A-B.

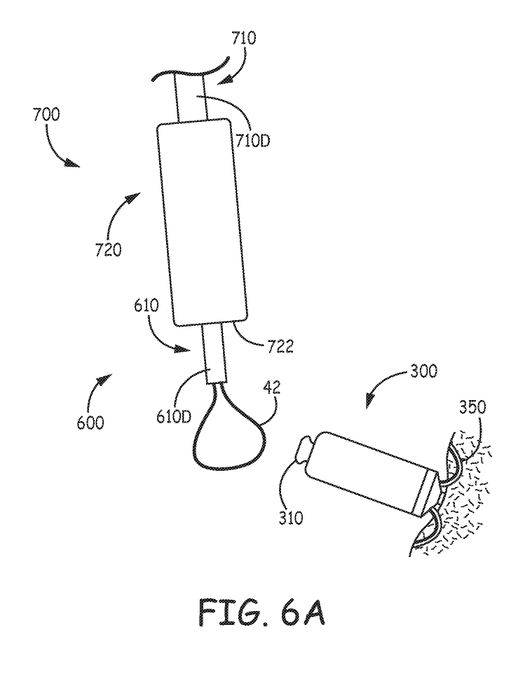

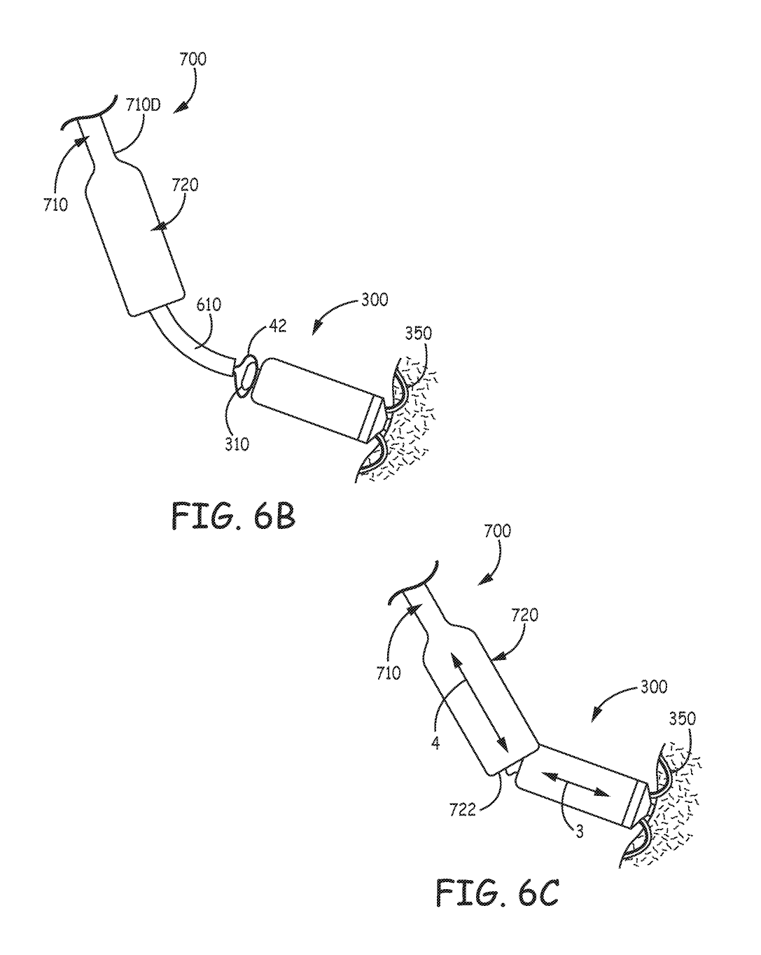

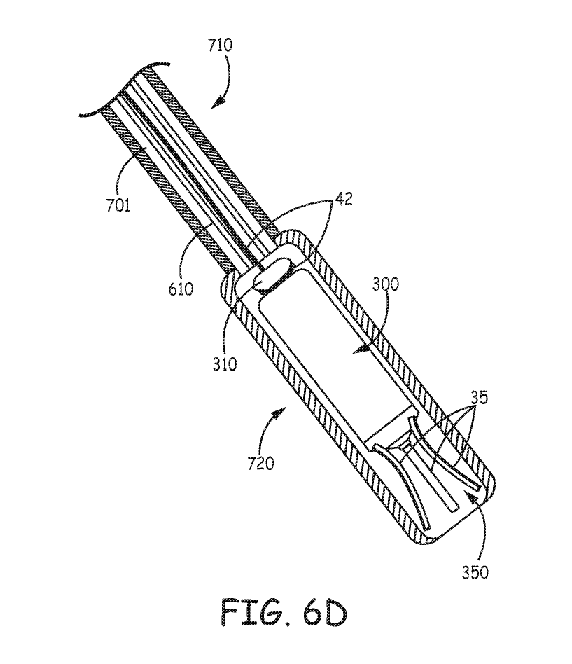

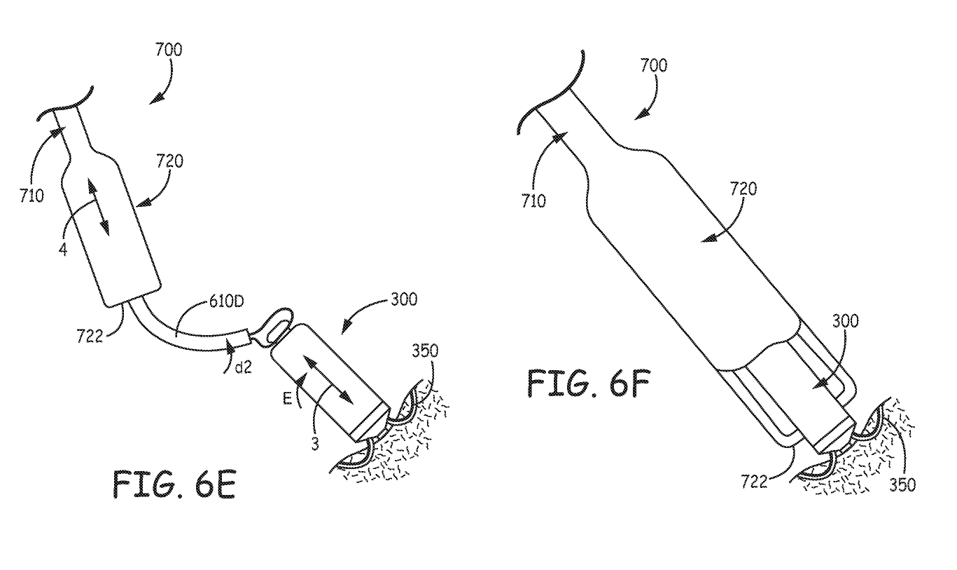

With further reference to FIG. 3, device fixation member 350 includes a plurality of fingers 35 spaced apart from one another around a perimeter of device housing distal end 382. Although only two fingers 35 of fixation member 350 are shown in FIG. 3, fixation member 350 may include as many as eight fingers 35. According to an exemplary embodiment, fixation fingers 35 are integrally formed with one another, having been cut from Nitinol tubing, according to methods known in the art. After cutting the Nitinol tubing, fingers 35 may be shaped by bending and holding fingers 35 in the illustrated curvature while heat treating, according to methods known to those skilled in the art. Fixation member 350 may be mounted to distal end 382 of device housing 380, for example, in a manner similar to that described for a fixation component 102 in co-pending and commonly assigned United States Patent Application 2012/0172690, which description is hereby incorporated by reference. The super-elastic nature of Nitinol allows fingers 35 to elastically deform between a relaxed condition, which is shown, and an extended condition, in which a free end 305 of each finger extends distally away from distal end 382 of device housing 380, for example, as shown in FIG. 6D.

FIG. 4A is a plan view of a catheter 400 and a device retrieval tool 40 that may be used together in an interventional medical system, according to some embodiments and methods. Catheter 400 is shown including an elongate shaft 410 and a device receptacle 420, wherein shaft 410 includes a sidewall 412 that defines a longitudinally extending lumen 401 (FIG. 4C) with a pre-formed bend along a distal portion 412d of the shaft sidewall. FIG. 4A illustrates receptacle 420 being coupled to a distal end 402 of sidewall distal portion 412d and including a distal opening 422 (also seen in FIGS. 4B-C), wherein receptacle 420 is in fluid communication with lumen 401. FIG. 4A further illustrates catheter shaft 401 including a passageway 414 formed through a proximal portion 412p of the shaft sidewall. According to the illustrated embodiment, passageway 414 is configured to allow passage of retrieval tool 40 therethrough and into lumen 401, without having to pass tool 40 through an additional length of lumen 401 within a handle assembly 450 of catheter 400. Thus, the location of passageway 414 may increase the ease by which an operator advances retrieval tool 40 out through distal opening 422 of receptacle 420, as shown in FIG. 4B, and by which the operator manipulates tool 40 to retrieve a medical device from an implant site, for example, device 300.

FIGS. 4A-B illustrate retrieval tool 40 including a snare member 42, for example, formed from a Nitinol wire, extending within an elongate shaft 41 of retrieval tool 40, for example, being slideably engaged therewith to open and close a distal loop thereof. Snare member 42 may include a gripper 44 secured around the wire thereof, for example, as shown in FIGS. 4A-B. Retrieval tool shaft 41 may be considered an inner shaft of the system, and catheter shaft 410 an outer shaft of the system. A pull band 14 and an actuator 54 of a steering assembly of retrieval tool 40 are also shown. Although not shown, those skilled in the art will understand that the steering assembly of tool 40 also includes an elongate deflection wire (e.g., stainless steel wire) that extends along a length of shaft 41, wherein a distal end thereof is coupled to band 14, and a proximal end thereof is coupled to actuator 54, so that when the operator rotates actuator 54, per arrow r, the pull wire deflects tool 40.

FIG. 4C is plan view, with an enlarged detail, and with cut-away cross-sections, of catheter 400, according to some embodiments. FIG. 4C illustrates a steering assembly of catheter 400 including a pull band 461, which is mounted to distal end 402 of shaft sidewall distal portion 412d, an actuator 456, which is mounted around shaft sidewall proximal portion 412p, and an elongate pull wire 46, for example, formed from a medical grade stainless steel wire having a diameter of approximately 0.010 inch, which extends distally from actuator 456 and within shaft lumen 401 to a distal end thereof, which is coupled to pull band 461. FIGS. 4A-C further illustrate actuator 456 being integrated into the aforementioned handle assembly 450 of catheter 400, which may also include a proximal port opening 407 in fluid communication with a proximal terminal opening 401PT of shaft lumen 401. Handle assembly 450 may be formed by opposing shell portions, for example, being injection molded from a relatively rigid medical grade plastic, such as Acrylonitrile butadiene styrene (ABS), according to methods known in the art; and, according to some embodiments, shaft wall proximal portion 412p is secured within the shell of handle assembly 450 by bonding, for example, with a biocompatible UV-cure adhesive. In FIG. 4C, catheter 400 is shown including a flushing assembly 470, which includes a tubular member 471 and a stopcock-type valve 474, and which is coupled to proximal terminal opening 401PT of shaft lumen 401 by port opening 407.

FIG. 4C further illustrates catheter shaft 401 including a pull wire opening 416 formed through shaft sidewall proximal portion 412p and spaced proximally from passageway 414, wherein a proximal end of pull wire 46 extends out from shaft lumen 401, through opening 416, and is coupled to a shank 64 of an interface component 462 of the steering assembly, for example, via a stainless steel hypo-tube crimped around the proximal end of wire 46 and interlocked with shank 64. According to the illustrated embodiment, interface component 462 is slideably mounted around shaft sidewall proximal portion 412p, and includes a flange 62 that engages with an internal thread 406 of actuator 456, so that rotation of actuator 456, per arrow R, causes interface component 462 to move longitudinally, thereby pulling wire 46 to deflect distal end 402 of shaft 410, per arrow D. Actuator 456 and interface component 462 may, like the aforementioned shell of handle assembly 450, be formed, for example, by injection molding, from a relatively rigid medical grade plastic known in the art, such as ABS.

With further reference to FIG. 4C, passageway 414 is spaced distally from actuator 456 by a distance S, which is no more than approximately 3-3.5 centimeters so that a handle 45 of retrieval tool 40 may be operated in proximity to handle assembly 450 of catheter 40, for example, facilitating simultaneous manipulation of both steering assembly actuators 54, 456 by a single operator. Furthermore, passageway 414 is located on a first side of lumen 401 that corresponds to an outside perimeter O of the pre-formed bend thereof, whereas pull wire opening 416 is located on a second side of lumen 401, opposite the first side and corresponding to an inside perimeter I of the pre-formed bend thereof. Thus, pull wire 46 and retrieval tool 40 may extend side-by-side in lumen 401 without significant risk of interfering with one another.

With reference back to FIG. 4B, snare member 42 of retrieval tool 40 has been advanced distally out from shaft 41 of retrieval tool 40 such that the loop thereof is open. Then, according to some methods, the operator can rotate actuator 54, per arrow r, to deflect a distal portion of shaft 41, for example, as indicated with the dashed lines in FIG. 4B, and thereby bring the loop of snare member 42 around device attachment feature 310. In some cases, the operator may need to twist, or torque, catheter shaft 410 while deflecting catheter shaft distal end 402, via actuator 456, in conjunction with manipulating retrieval tool 40, to bring the loop of snare member 42 around an implanted device, for example, around attachment feature 310 of device 300 (FIG. 3). Once the loop extends around device attachment feature 310, the operator may retract snare member 42 relative to shaft 41, to constrict the loop, advance receptacle 420 of catheter 400 around the snared device 300, and apply a pull force to retrieval tool 40 to disengage device fixation member 350 from the implant site.

A perimeter of passageway 414 is preferably configured for sealing engagement around shaft 41 of retrieval tool 40 as tool 40 is passed therethrough, for example, having an overlay of an elastic polymer, such as medical grade silicone rubber. In some embodiments, the perimeter of passageway may also be reinforced by a polymer tubing welded to sidewall proximal portion 412p. The enlarged detail view shown in the dashed-line box of FIG. 4C illustrates an embodiment of passageway 414 in which an elastic slit valve (e.g., medical grade silicone rubber) forms the sealing perimeter thereof. Opposing edges of the slit valve confront one another to substantially seal passageway 414 against significant backflow, for example, to provide haemostasis when retrieval tool 40 is not passed therethrough, but spread apart, per arrows of the detail, to allow passage of retrieval tool 40 therebetween.

According to an exemplary embodiment, sidewall 412 of catheter shaft 410, for example, extending over a length of approximately 100 centimeters, may be formed by a medical grade polymer, such as one or more appropriate grades of polyether block amide, which are arranged for decreasing stiffness from handle assembly 450 to shaft distal end 402 (e.g., PEBAX® 3533, 6333, 4033, and 7233), and which may or may not be reinforced with a stainless steel braid that may be of a variable pic rate. Alternate shaft sidewall constructions, for example, incorporating a polymer of a single durometer along the length thereof, and/or incorporating a laser cut hypo-tube of a pattern that increases in flexibility from shaft sidewall proximal portion 412p to shaft sidewall distal portion 412d are not outside the scope of the present invention. Receptacle 420, in some exemplary embodiments, may be formed from a medical grade polyether block amide (e.g., PEBAX® 7233 SA-01), and preferably includes a radiopaque marker band 425 (FIGS. 4A-B) integrated therein. According to some embodiments, marker band 425 is formed from a Tungsten filled polymer, for example, 75% Tungsten and 25% Vestamid® L2140, which is heat bonded to receptacle 420, for example, while being secured thereto with a sacrificial heat-shrink tube.

According to some alternate embodiments, marker band 425 is a gold foil, for example, having a thickness of approximately ten microns, which is secured around receptacle 420 by a reflow of the material thereof thereover. In yet further embodiments, a radiopaque filler, such as Tungsten, may be blended with the aforementioned PEBAX® material prior to extruding receptacle 420. Catheter shaft lumen 401 may have a diameter of approximately 0.154 inch (3.9 mm), passageway 414 through shaft proximal portion 412p may have a diameter of approximately 0.12 inch (3 mm), and catheter receptacle 420 may have an internal diameter of approximately 0.296 inch (7.5 mm) and extend over a length of approximately 1.3 inches (33 mm) to contain a medical device, such as device 300 of FIG. 3. It should be noted that catheter 400, in some cases, may also be employed to deliver device 300 to the implant site.

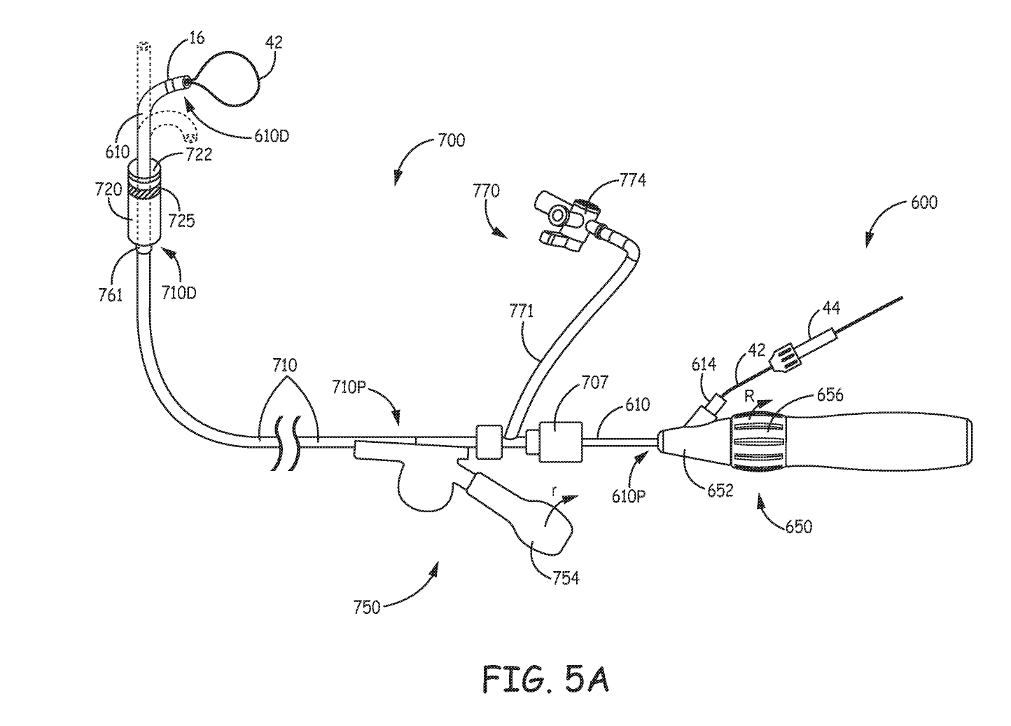

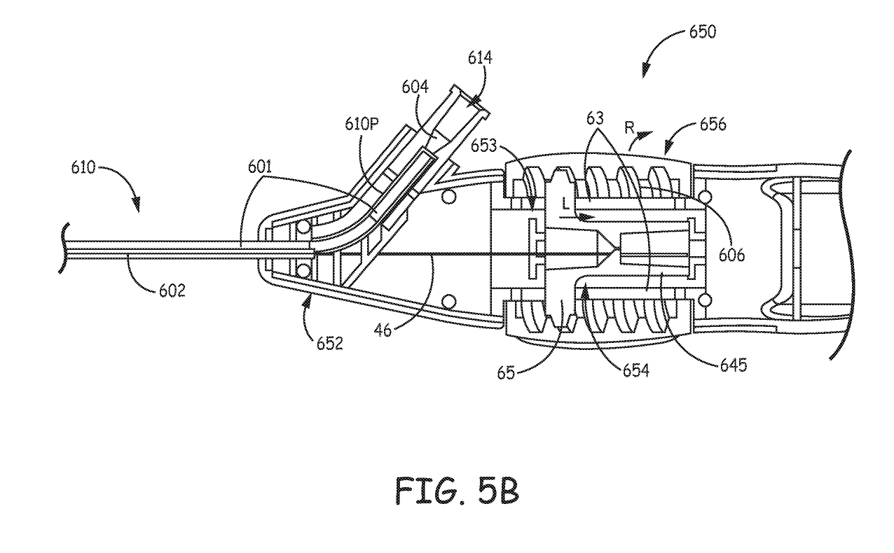

FIG. 5A is a plan view of an interventional medical system, according to some alternate embodiments, which includes a retrieval tool 600 and a catheter 700; and FIG. 5B is a cross-section view through a portion of retrieval tool 600, according to some embodiments. FIGS. 5A-B illustrate retrieval tool 600 including an elongate shaft 610, a steering assembly 650 coupled to shaft 610, the above described snare member 42 extending within a first longitudinally extending lumen 601 of shaft 610, and a passageway 614 that allows passage of snare member 42 into lumen 601. FIGS. 5A-B further illustrate steering assembly 650 including an actuator 656 mounted in proximity to a proximal end 610P of shaft 610, wherein the above described pull wire 46 extends distally from actuator 656 and within a second lumen 602 of shaft 610; steering assembly further includes a pull band 16 coupled to a distal end 610D of shaft 610 and to pull wire 46. Passageway 614 is shown being located distal to steering assembly actuator 656, and is preferably spaced distally from actuator 656 by no more that approximately 3 centimeters to 3.5 centimeters, for example, to increase the ease by which an operator can both move snare member 42, by grasping gripper 44, and manipulate steering assembly actuator 656 to deflect shaft 610.

With reference to FIG. 5B, in some preferred embodiments, passageway 614 includes an elastic sealing perimeter 604, for example, having an overlay of an elastic polymer, such as medical grade silicone rubber, that provides sealing engagement around snare member 42 when passed therethrough. In some embodiments, sealing perimeter 604 of passageway 614 may be formed by an elastic slit valve (e.g., medical grade silicone rubber), like that described above in conjunction with FIG. 4C, so that sealing perimeter 604 can provide haemostasis when snare member 42 is not passed therethrough.

FIG. 5B further illustrates steering assembly 650 including a tapered member 652 and a core 656, wherein tapered member 652 couples shaft proximal end 610P to core 656, and wherein passageway 614 is formed in tapered member 652. The illustrated steering assembly 650 also includes an interface component 654 slideably mounted to core 656, wherein core 656 is shown having opposing slots 63 formed through a sidewall thereof with opposing flanges 65 of interface component 654 projecting therethrough to engage with an internal thread 606 of actuator 656. With further reference to FIG. 5B, interface component 654 further includes a shank 645 coupled to pull wire 46. According to the illustrated embodiment, actuator thread 606 is rotatable, per arrow R, around core 654 to move interface component 654 longitudinally, per arrow L, and thereby move pull wire 46 to deflect shaft distal end 610D, for example, as indicated with dashed lines in FIG. 5A. It should be noted that an alternate embodiment of retrieval tool 600 may include a sliding actuator, rather than a rotating actuator, in a different form of a steering assembly, for example, patterned after the deflection assembly described in a co-pending and commonly assigned U.S. patent application having the Ser. No. 14/694,579 (filed on Apr. 23, 2015), which is hereby incorporated by reference. Embodiments of the deflection assembly described in the '579 application include a hub 360 that forms a core of the deflection assembly to which a slider component 350, which acts as an actuator, is mounted, wherein a strain relief element 328, which is tapered like tapered member 652, couples a catheter shaft 310 to hub 360. Thus, element 328 of the '579 application may be configured to include a passageway, like passageway 614, for insertion of a snare member into the lumen of shaft 310, according to some alternate embodiments of the instant invention.

Steering assembly actuator 656, core 653, interface component 654, and tapered member 652 may each be formed, for example, by injection molding, from a relatively rigid medical grade plastic known in the art, such as ABS; and tapered member 652 may include an overlay of a more flexible material, such as a medical grade thermoplastic elastomer like Santoprene™ or Medalist®, in some embodiments. Shaft 610 may be formed by an extruded dual lumen tube overlaid by a stainless steel braid-reinforced polymer jacket, wherein a stiffness of shaft 610 decreases along a length thereof, from proximal end 610P to distal end 610D, for example, by varying a thickness of the polymer jacket, and/or the durometer of the polymer jacket, and/or the pic rate of the stainless steel braid reinforcement. According to an exemplary embodiment, the dual lumen tube of shaft 610 is a medical grade polyether block amide (e.g., PEBAX® 7233 or 5533), the polymer jacket is PEBAX® 7233 fused to the stainless steel braid, and each lumen of the dual lumen tube is lined with a lubricious material, such as fluoropolymer (e.g., PTFE), or high or low density polyethylene.

With reference back to FIG. 5A, catheter 700 includes an elongate shaft 710 in which retrieval tool shaft 610 is received in sliding engagement; thus, catheter shaft 710 may be said to be an outer shaft of the system, and retrieval tool shaft 610 an inner shaft of the system. FIG. 5A illustrates shaft 710 being coupled to a handle assembly 750, at a proximal end 710P thereof, and a receptacle 720 joined to a distal end 710D thereof. FIG. 5A further illustrates handle assembly 750 including a proximal port opening 707, which may be formed by an adjustable haemostasis valve (e.g., a Tuohy-Borst type valve), and which is in fluid communication with a longitudinally extending lumen 701 (FIG. 6D) of outer shaft 710, to allow passage of inner shaft 610 therethrough. A flushing assembly 770, which includes a tubular member 771 and a stopcock-type valve 774, is also shown coupled to handle assembly 750, wherein tubular member 771 is in fluid communication with the lumen of outer shaft 710.

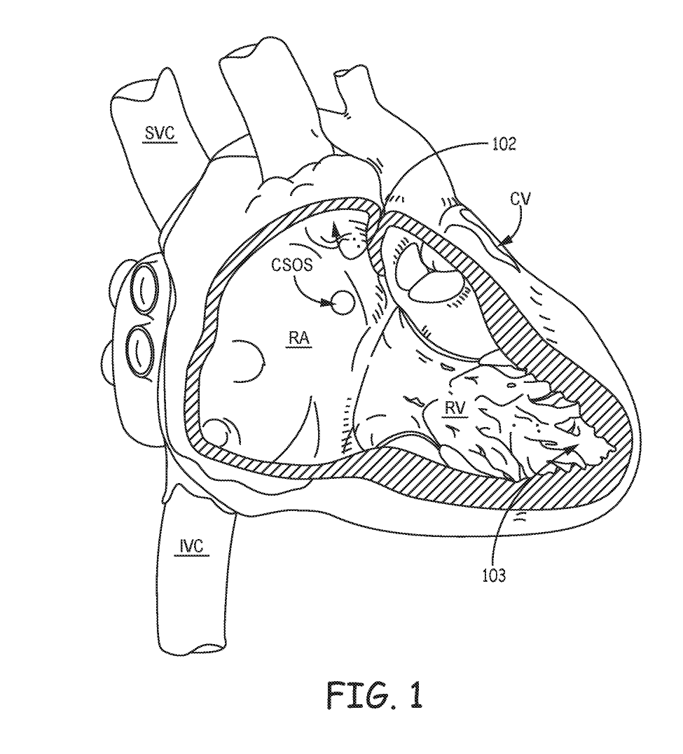

According to the illustrated embodiment, receptacle 720 of catheter 700 is sized to contain an implantable medical device therein (e.g., device 300), and is in fluid communication with the lumen of shaft 710. Receptacle 720 is shown including a distal opening 722, which is sized to allow passage therethrough of shaft 610 and snare member 42 of retrieval tool 600, as well as passage of device 300. Handle assembly 750 further includes an actuator 754 of a steering assembly of catheter 700, wherein the steering assembly is coupled to shaft 710, for example, via a pull wire (not shown) and a pull band 761, which is shown mounted to distal end 710D of shaft 710. A general construction of shaft 710 and receptacle 720 may be similar to that described above for the exemplary embodiment of shaft 410 and receptacle 420 of catheter 400. According to some methods, an operator may position receptacle 720 in proximity to an implant site, for example, in the patient's right ventricle RV (FIG. 2), by advancing catheter 700 into the right heart through the inferior vena cava IVC, for example, from a femoral vein access site and through an introducer sheath (e.g., like sheath 500 of FIG. 4B), and then by maneuvering shaft 710, via steering assembly actuator 754, to cross the tricuspid valve TV. After the positioning receptacle 720 in proximity to the implant site, the operator may advance shaft 610 and snare member 42 of retrieval tool 600 out from distal opening 722 of receptacle 720 and then retrieve device 300, for example, according to the steps described below in conjunction with the schematics FIGS. 6A-F. It should be noted that method steps described below can be employed to retrieve an implanted device from any of the cardiac implant sites shown in FIG. 1.

FIG. 6A illustrates snare member 42, which has been inserted into lumen 601 of shaft 610, via passageway 614 (FIG. 5B), and advanced distally out from a distal opening of lumen 601 at distal end 610D of shaft 610, such that the loop of snare member 42 is open and positioned in proximity to attachment feature 310 of device 300. Then, according to some methods, the operator manipulates steering assembly actuator 656 of retrieval tool 600, as described above, to deflect shaft 610, for example, as represented with the dashed lines in FIG. 5A, and thereby bring the loop of snare member 42 around attachment feature 310, as shown in FIG. 6B. Once the loop is positioned as shown, the operator may retract snare member 42, via gripper 44, with respect to shaft 610 to constrict the loop around device attachment feature 310, and thereby snare device 300. In some cases, to snare device 300, the operator may also need to twist, or torque, catheter shaft 710 and/or deflect catheter shaft 710, via actuator 754, and/or further maneuver retrieval tool 600, which may also involve moving snare member 42 back and forth with respect to shaft 610, via gripper 44. With further reference to FIG. 5A, note again the proximity of passageway 614 to actuator 656, which may increase the ease by which the operator can both move snare member 42, by grasping gripper 44, and manipulate steering assembly actuator 656 to deflect shaft 610 in the maneuvering of tool 600.

Next, the operator may advance catheter 700 relative to retrieval tool 600 to position distal opening 722 of receptacle 720 around the snared medical device 300, as shown in FIG. 6C, before applying a pull force through the retracted snare member 42, which disengages device fixation member 350 from the implant site and brings device 300 into receptacle 720, for example, as shown in FIG. 6D. According to some preferred methods, prior to disengaging device fixation member 350, the operator brings a significant portion of device 300 into receptacle 720, but, with further reference to FIG. 6C, a misalignment between a longitudinal axis 4 of receptacle 720 and axis 3 of device 300 may hinder the operator from doing so. Thus, in some methods, and with reference to FIG. 6E, the operator, after snaring device 300, may again manipulate retrieval tool 600, via steering assembly actuator 656, to further deflect distal end 610D of shaft 610, per arrow d2, and thereby reorient device 300, per arrow E, so that axes 3 and 4 are better aligned to advance receptacle 720 over device 300. According to some methods, the operator advances catheter receptacle 720 over device 300 until receptacle distal opening 722 abuts the implant site, as shown in FIG. 6F, prior to disengaging device fixation member 350 from the implant site by applying the pull force through snare member 42.

In the foregoing detailed description, the invention has been described with reference to specific embodiments. However, it may be appreciated that various modifications and changes can be made without departing from the scope of the invention as set forth in the appended claims.

We claim:

1. An interventional medical system comprising an elongate shaft, a steering assembly coupled to the shaft, a snare member, and a passageway configured to allow passage of the snare member therethrough and into a first longitudinally extending lumen of the shaft, the steering assembly including an actuator mounted in proximity to a proximal end of the shaft, an elongate pull wire extending distally from the actuator, within either the first lumen or a second longitudinally extending lumen of the shaft, and a pull band being coupled to a distal end of the shaft and to the pull wire; and wherein an improvement to the system comprises:
the passageway being spaced distally from the steering assembly actuator by no more than approximately 3 to 3.5 centimeters; and
the passageway including an elastic sealing perimeter.

2. The system of claim 1, wherein the elastic sealing perimeter of the passageway provides haemostasis when the snare member is not passed therethrough.

3. The system of claim 1, wherein the steering assembly further comprises a core, around which the steering assembly actuator is mounted, and a tapered member that couples the proximal end of the shaft to the core; and wherein the passageway is formed in the steering assembly tapered member.

4. The system of claim 3, wherein:
the steering assembly actuator comprises an internal thread rotatable about the steering assembly core; and
the steering assembly further comprises an interface component slideably mounted to the steering assembly core, the interface component including a shank and a flange, the shank being coupled to the pull wire, and the flange being engaged with the internal thread of the actuator such that rotating the actuator thread moves the interface component longitudinally.

5. The system of claim 1, further comprising:
an elongate outer shaft having a longitudinally extending lumen; and
wherein the elongate shaft of claim 1 comprises an inner shaft, the lumen of the outer shaft being configured to receive the inner shaft in sliding engagement therein;
the outer shaft has a receptacle joined to a distal end thereof and being in fluid communication with the lumen of the outer shaft; and
the receptacle is sized to contain an implantable medical device therein, and has a distal opening sized to allow passage therethrough of the inner shaft, the snare member, and the device.

6. The system of claim 5, further comprising another steering assembly, the other steering assembly being coupled to the outer shaft.

7. The system of claim 1, further comprising:
an elongate inner shaft having a longitudinally extending lumen that receives the snare member in sliding engagement therein, the passageway being configured to allow passage of the inner shaft therethrough; and
wherein the elongate shaft of claim 1 comprises an outer shaft that has a receptacle joined to a distal end thereof, the receptacle being in fluid communication with the lumen of the outer shaft; and
the receptacle is sized to contain an implantable medical device therein, and has a distal opening sized to allow passage of the inner shaft, the snare member, and the device therethrough.

8. The system of claim 7, further comprising another steering assembly, the other steering assembly being coupled to the inner shaft.

9. An interventional medical system comprising an implantable medical device, a retrieval tool, and a catheter; the medical device comprising an electronic controller, a hermetically sealed housing containing the controller, an electrode electrically coupled to the controller and mounted in proximity to a distal end of the housing, an attachment feature joined to a proximal end of the housing, and a fixation member mounted to the distal end of the housing; the retrieval tool configured to snare the attachment feature of the medical device; and the catheter comprising an elongate shaft, a receptacle, and a steering assembly; the catheter shaft including a sidewall that defines a longitudinally extending lumen of the shaft with a pre-formed bend along a distal portion of the sidewall; the catheter receptacle being coupled to a distal end of the distal portion of the sidewall, the catheter receptacle being in fluid communication with the shaft lumen, sized to contain the medical device, and having a distal opening sized to receive passage of the device therethrough; the catheter steering assembly including an actuator mounted around a proximal portion of the shaft sidewall, an elongate pull wire extending distally from the actuator and within the lumen of the shaft, and a pull band coupled to a distal end of the pull wire and mounted to the distal end of the shaft sidewall distal portion; and wherein an improvement to the catheter shaft comprises:
a passageway formed through a proximal portion of the shaft sidewall on a first side of the shaft lumen, the first side of the lumen corresponding to an outside perimeter of the pre-formed bend thereof; and
wherein the passageway is spaced distally from the actuator of the steering assembly by no more than approximately 3 to 3.5 centimeters;
the passageway is sized to allow passage of the retrieval tool therethrough and into the shaft lumen; and
a perimeter of the passageway is configured for sealing engagement around the retrieval tool as the tool is passed therethrough.

10. The system of claim 9, wherein the catheter shaft further comprises a pull wire opening formed through the proximal portion of the shaft sidewall on a second side of the shaft lumen, the second side of the lumen being opposite the first side of the lumen and corresponding to an inside of the pre-formed bend thereof, the pull wire opening being spaced proximally from the port, and a proximal end of the pull wire extending through the pull wire opening from the shaft lumen to the actuator of the steering assembly.

11. The system of claim 10, wherein the catheter further comprises a flushing assembly; and the shaft lumen extends proximally from the pull wire opening to a proximal terminal opening thereof, the proximal terminal opening being coupled to the flushing assembly.

12. The system of claim 10, wherein:
the actuator of the catheter steering assembly comprises an internal thread, the actuator being mounted around the proximal portion of the shaft sidewall such that the internal thread is rotatable thereabout; and
the steering assembly further comprises an interface component slideably mounted around the proximal portion of the shaft sidewall, the interface component including a shank and a flange, the shank being coupled to the proximal end of the pull wire, and the flange being engaged with the internal thread of the actuator such that rotating the actuator causes the thread thereof to move the interface component longitudinally.

13. The system of claim 9, wherein the perimeter around the passageway of the catheter shaft is elastic to substantially seal the passageway against significant backflow, to provide haemostasis when the retrieval tool is not passed therethrough.

14. A method for retrieving an implantable medical device from an implant site in a patient, the medical device comprising an electronic controller, a hermetically sealed housing containing the controller, an electrode electrically coupled to the controller and mounted in proximity to a distal end of the housing, an attachment feature joined to a proximal end of the housing, a longitudinal axis extending between the proximal and distal ends of the housing, and a fixation member mounted to the distal end of the housing, and the method comprising:
inserting a snare member into a longitudinally extending lumen of an elongate shaft through a passageway formed in a tapered member of a steering assembly that is coupled to the shaft, the passageway being spaced distally from an actuator of the steering assembly of the system by no more than approximately 3 to 3.5 centimeters;
advancing the shaft with the snare member inserted therein out from a distal opening of a receptacle of a catheter, the receptacle being positioned in proximity to the implant site;
advancing the snare member out through a distal opening of the lumen of the advanced shaft, so that a loop thereof is opened in close proximity to the attachment feature of the device at the implant site;
manipulating the steering assembly actuator to deflect the shaft and thereby bring the loop of the advanced snare member around the attachment feature of the medical device;
retracting the snare member with respect to the shaft to constrict the loop around the device attachment feature and thereby snare the device;
advancing the receptacle of the catheter relative to the shaft to position the distal opening of the receptacle around the snared device;
applying a pull force through the retracted snare member to disengage the fixation member of the device from the implant site.

15. The method of claim 14, further comprising manipulating the steering assembly actuator to deflect the shaft, after snaring the device, thereby approximately aligning a longitudinal axis of the device with a longitudinal axis of the receptacle of the catheter.

16. The method of claim 14, further comprising abutting the distal opening of the receptacle of the catheter against the implant site, after positioning the distal opening of the receptacle around the snared device, and before applying the pull force through the snare member.
